(19) United States
(12) Patent Application Publication
(10) Pub. No.: US 2025/0381022 A1
(43) Pub. Date: Dec. 18, 2025

(54) URETHRAL TREATMENT APPARATUS AND METHOD

(71) Applicant: ProArc Medical Ltd., Yokne'am Illit (IL)

(72) Inventors: Gilad Hizkiyahu, Givat-Ela (IL); Liran Ankri, Atlit (IL); Dean Oron, Herzliya (IL)

(21) Appl. No.: 18/876,460

(22) PCT Filed: Jun. 30, 2023
(86) PCT No.: PCT/IB2023/056813

§ 371 Date: Dec. 18, 2024

Related U.S. Application Data
(60) Provisional application No. 63/357,122, filed on Jun. 30, 2022.
(60) Provisional application No. 63/430,030, filed on Dec. 4, 2022.

(51) Int. Cl. A61F 2/02; A61B 18/00; A61B 18/14; A61B 90/00

(52) U.S. Cl. CPC A61F 2/02; A61B 18/1485; A61B 90/361; A61B 2018/00083; A61B 2018/00184; A61B 2018/0022; A61B 2018/00517; A61B 2018/00601; A61B 2018/1435; A61F 2230/0004

(57) ABSTRACT

Apparatus (20) for treatment of a constricted urethra (90), is provided. A delivery tool (30) is advanced to a location in an area of the urethra that is to be treated (92). A distal portion (34) of the delivery tool includes an expandable element (50) that is expanded within the urethra, an implant (70) disposed outside the outer surface of the expandable element, and a cutting electrode (60) releasably coupled to an outer surface of the implant and configured to apply electrical current to tissue of the urethra to form an implant-receiving cut in tissue surrounding the lumen of the urethra by the application of the electrical current. The cutting electrode is shaped to conform with the shape of the implant such that the implant-receiving cut is formed in the shape of the implant substantially without movement, e.g., rotation of the cutting electrode. Other applications are also described.

### CROSS-REFERENCES TO RELATED APPLICATIONS

The present application claims priority from U.S. Provisional Patent Application No. 63/357,122 to Hizkiyahu et al., filed Jun. 30, 2022, and from U.S. Provisional Patent Application No. 63/430,030 to Hizkiyahu et al., filed Dec. 4, 2022, both entitled “URETHRAL TREATMENT APPARATUS AND METHOD”, and both of which are incorporated herein by reference.

### FIELD OF EMBODIMENTS OF THE INVENTION

Some applications of the present invention generally relate to devices and methods for treatment of tissue. More particularly, some applications of the present invention relate to devices and methods for treatment of benign prostatic hyperplasia (BPH).

### BACKGROUND

Benign prostatic hyperplasia (BPH), prostate gland enlargement, is a common condition as men age. BPH is a non-cancerous condition in which the prostate enlarges to the point where it constricts the urethra and impedes the flow of urine, making urination difficult and painful, and in extreme cases completely impossible.

### SUMMARY

In some applications of the present invention, apparatus and methods are provided for treatment of a constricted bodily lumen, for example a urethra that is constricted due to benign prostatic hyperplasia (BPH). Typically, the apparatus includes a delivery tool, which has a proximal portion and a distal portion. The distal portion of the delivery tool is configured to be advanced to a location in an area of the urethra that is to be treated due to enlargement of the prostate and consequent constriction of the area. In some applications, the distal portion includes an operational head having an expandable element, an implant, and a cutting electrode. The expandable element e.g., an inflatable balloon, expands within the urethra such as to enlarge the urethra and dilate the constricted area. The cutting electrode is typically releasably coupled to the implant and forms a cut in an inner surface of the urethra, subsequently to the expandable element having expanded in the urethra. The cutting electrode is typically shaped to conform with a shape of the implant (and is typically disposed on the outside of the implant) such that the implant-receiving cut is formed in the shape of the implant substantially without movement, e.g., without rotation, of the cutting electrode. Subsequently to the cutting electrode forming the cut in the urethra, the implant is released into the cut within the urethra and implanted in tissue of the prostate surrounding the cut in the urethra, to maintain the urethra in a dilated state.

There is therefore provided, in accordance with some applications of the present invention, apparatus for treatment of a urethra, a lumen of which is constricted, the apparatus including:
a delivery tool having a proximal portion and a distal portion, the distal portion configured to be advanced to a location in an area of the urethra that is to be treated, the distal portion including:
   an expandable element that is configured to be expanded within the urethra such as to enlarge the urethra in the area to be treated, the expandable element defining an outer surface;
   an implant that is disposed outside the outer surface of the expandable element; and
   a cutting electrode releasably coupled to an outer surface of the implant and configured to apply electrical current to tissue of the urethra to form an implant-receiving cut in tissue surrounding the lumen of the urethra by the application of the electrical current,
   the cutting electrode being shaped to conform with a shape of the implant such that the implant-receiving cut is formed in the shape of the implant substantially without movement of the cutting electrode.

In some applications, the cutting electrode is shaped to conform with a shape of the implant such that the implant-receiving cut is formed in the shape of the implant substantially without rotation of the cutting electrode.

In some applications, the cutting electrode is configured to be retracted from the urethra subsequently to forming the implant-receiving cut.

In some applications, the cutting electrode has a distal portion that is releasably coupled to the implant and a proximal extension that is disposed in the delivery tool, and the cutting electrode is configured to be retracted from the urethra by pulling the proximal extension of the electrode in a proximal direction.

In some applications, the implant is configured as a C-shaped open ring.

In some applications, the implant is configured to be wound around the expandable element, while the distal portion of the delivery tool is advanced to the location in an area of the urethra that is to be treated.

In some applications, the implant has a thickness of 0.01-1 mm.

In some applications, the implant has a width of 0.5-4 mm.

In some applications, the cutting electrode is formed of nitinol.

In some applications, the implant is electrically insulated from the cutting electrode.

In some applications, the implant is configured to maintain the urethra in a dilated state in the area of the cut by being deployed in the cut.

In some applications, the apparatus further includes an imaging device disposed in the delivery tool and configured to provide visualization of the area of the urethra that is treated by the imaging device.

In some applications, the expandable element includes an inflatable balloon.

In some applications, the inflatable balloon is transparent.

In some applications, the apparatus further includes an imaging device disposed in the delivery tool, and the inflatable balloon is configured to provide visualization of the area of the urethra that is treated by the imaging device, via the inflatable balloon.

In some applications, the apparatus further includes one or more sutures configured to releasably couple the cutting electrode to the implant prior to application of the electrical current by the cutting electrode.

In some applications, the one or more sutures are configured to become decoupled from the implant by the application of the electrical current, to thereby release the cutting electrode from the implant.

In some applications, the cutting electrode includes coiled tips at each end of the cutting electrode, each one of the coiled tips holds a respective end of the cutting electrode in place with respect to the implant by preventing the end of the cutting electrode from passing through one of the sutures that is disposed closest to that end of the implant, and in response to the cutting electrode being pulled proximally, the coiled tip is configured to straighten and pass through the one or more sutures.

In some applications, the apparatus further includes a single implant carrier arm extending from a shaft of the delivery tool and being disposed outside the outer surface of the expandable element, the implant carrier being configured to release the implant into the implant receiving cut subsequently to the cutting electrode forming the implant-receiving cut.

In some applications, the implant carrier arm is coated with an electrically insulating coating.

In some applications, the apparatus further includes an electrically-non-conductive implant holder coupled to the implant carrier arm, and:
the implant is configured to be disposed between the implant carrier arm and the implant holder such that the implant is held in place by the implant carrier arm and the implant holder; and
the implant holder being configured to be retracted proximally with respect to the implant carrier arms such that the implant is released by the implant carrier arms into the implant-receiving cut.

In some applications, the apparatus further includes at least one radial arm having a length which is: (a) sufficient to apply pressure to the walls of the urethra to dilate the urethra in an area that is proximal to the inflatable element, when the inflatable element is in the inflatable state; and (b) not sufficient to contact the implant.

There is further provided, in accordance with some applications of the present invention, a method for treating a urethra, a lumen of which, is constricted due to benign prostatic hyperplasia (BPH) including:
identifying a constricted area of the urethra requiring treatment;
inserting into the urethra a delivery tool that includes:
   an expandable element that defines an outer surface,
   an implant disposed on the outer surface of the expandable element, and
   a cutting electrode releasably coupled to an outer surface of the implant;
using the delivery tool, delivering, to the identified constricted area of the urethra, the inflatable element;
expanding the urethra by expanding the expandable element in the identified constricted area of the urethra;
subsequently, forming an implant-receiving cut in the tissue surrounding the lumen of the urethra, by applying an electrical current to the tissue using the cutting electrode, the cutting electrode is shaped to conform with a shape of the implant such that the cutting electrode forms the implant-receiving cut in the shape of the implant substantially without the cutting electrode being moved; and releasing the implant into the cut to maintain the urethra in a dilated state.
In some applications, the method further includes retracting the cutting electrode from the urethra subsequently to forming the implant-receiving cut.

In some applications, the cutting electrode is shaped to conform with a shape of the implant such that the cutting electrode forms the implant-receiving cut in the shape of the implant substantially without the cutting electrode being rotated.

In some applications, prior to applying the electrical current to the tissue using the cutting electrode, the cutting electrode is coupled to the implant via one or more sutures, and applying the electrical current to the tissue using the cutting electrode includes releasing the cutting electrode form the implant by decoupling the sutures from the implant.

In some applications, the method further includes using radial arms, applying pressure to walls of the urethra in an area proximal to the expandable element such as to maintain the area of the urethra that is proximal to the expandable element in a dilated state.

The present invention will be more fully understood from the following detailed description of embodiments thereof, taken together with the drawings, in which:

### BRIEF DESCRIPTION OF THE DRAWINGS

FIGS. 1A and 1B, are schematic illustrations of an apparatus for treatment of a urethra that is constricted due to benign prostatic hyperplasia (BPH), in accordance with some applications of the present invention;

FIGS. 2A, 2B, and 2C are schematic illustrations of an operational head disposed at the distal portion of the apparatus, the operational head comprising an expandable element, an implant, and a cutting electrode, in accordance with some applications of the present invention;

FIGS. 3A, 3B, 3C, 3D, 3E, and 3F are schematic illustrations of the apparatus for treatment of the urethra being advanced and deployed in the urethra, in accordance with some applications of the present invention;

FIG. 4 is a flow chart showing steps in an exemplary method practiced in accordance with some applications of the present invention;

FIGS. 5A and 5B are schematic illustrations of components of the operational head disposed at the distal portion of the apparatus for treatment of a urethra that is constricted due to benign prostatic hyperplasia (BPH), in accordance with some applications of the present invention;

FIGS. 6A and 6B are photographs showing an example of a urethra of a subject, prior to, and following, treatment of the subject using the apparatus for treatment of a urethra that is constricted due to benign prostatic hyperplasia (BPH), in accordance with some applications of the present invention;

FIGS. 7A and 7B are photographs showing a contrast fluid being injected through a urethra of a subject following treatment of the subject using the apparatus for treatment of a urethra that is constricted due to benign prostatic hyperplasia (BPH), in accordance with some applications of the present invention; and

FIG. 8 and FIG. 9 are photographs showing the implant implanted in an enlarged prostate, in accordance with some applications of the present invention.

### DETAILED DESCRIPTION OF EMBODIMENTS

Some applications of the present invention, relate to apparatus and methods for treatment of intrabody lumens, and, more particularly, but not exclusively, to apparatus and methods for dilating and/or assisting in dilation and/or maintaining dilation of an intrabody lumen, e.g., the urethra, to relieve obstruction of the lumen.

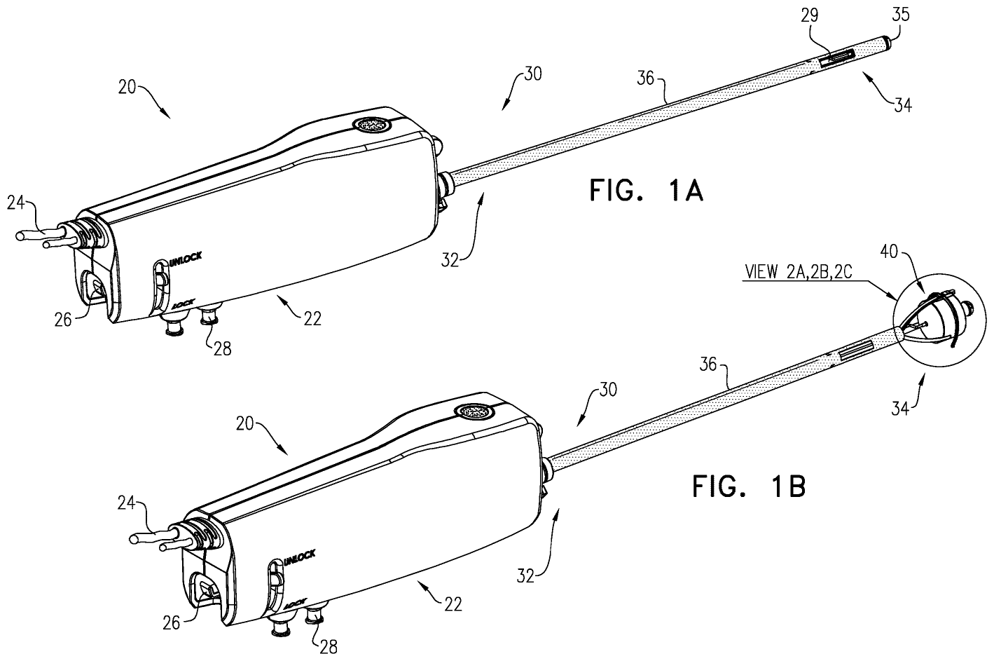

Reference is made to FIGS. 1A and 1B, which are schematic illustrations of an overview of apparatus 20 for treatment of a urethra that is constricted due to benign prostatic hyperplasia (BPH), in accordance with some applications of the present invention.

For some applications, apparatus 20 comprises a delivery tool 30, e.g., a transluminal catheter. Delivery tool 30 typically comprises a proximal portion 32 and a distal portion 34 and an elongated shaft 36 disposed between proximal and distal portions 32 and 34. Distal portion 34 is configured to be advanced distally in the urethra to a location in an area of the urethra that requires treatment due to being constricted. Typically, distal portion 34 is shaped to define a blunt atraumatic tip portion 35 as shown in FIGS. 1A-B. It is noted that the term “distal” and related terms, when used with reference to a device or a portion thereof, should be interpreted to mean an end of the device or the portion thereof that, when inserted into a subject's body, is typically further from the location through which the device is inserted into the subject's body. The term “proximal” and related terms, when used with reference to a device or a portion thereof, should be interpreted to mean an end of the device or the portion thereof that, when inserted into a subject's body, is typically closer to the location through which the device is inserted into the subject's body.

In some applications, apparatus 20 comprises a handle 22 configured to be held by a user, e.g., a surgeon. In some applications, handle 22 is connected to a power source (not shown) via a cable 24. Additionally, or alternatively, handle 22 comprises a power source included within. Typically, handle 22 includes levers and actuation mechanisms that are manually operated. As shown, typically, elongated shaft 36 is connected to handle 22. In some applications, apparatus 20 comprises an inlet and/or outlet 28, configured to allow insertion and removal of fluids from the apparatus 20, for example: air, water and/or saline.

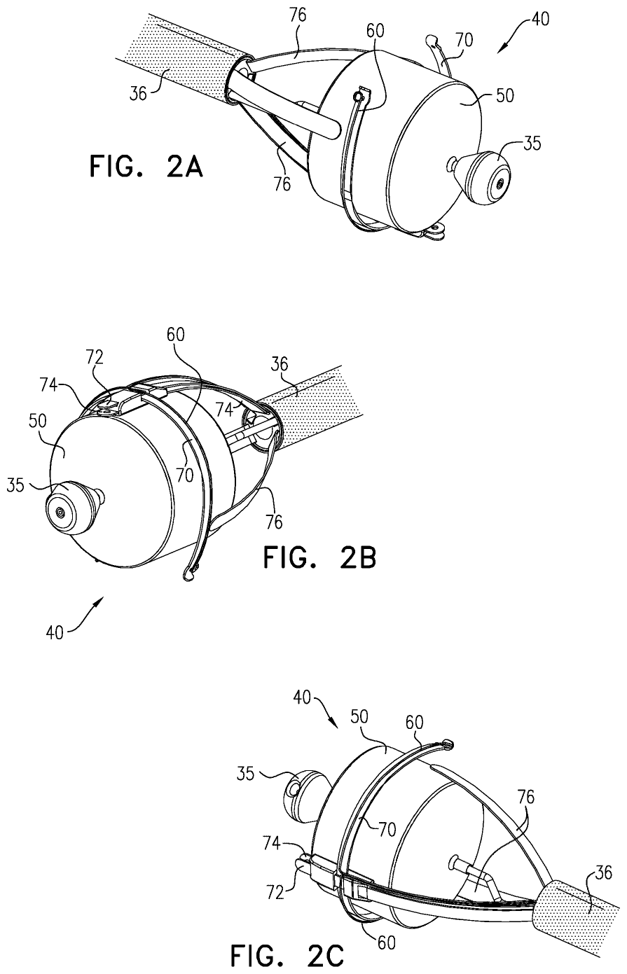

Typically, apparatus 20 further comprises an operational head 40, as shown for example in FIG. 1B, which is shown in an expanded configuration. Operational head 40 is typically advanced distally in delivery tool 30 in a radially collapsed state and is radially expanded in the urethra in the area to be treated, as will be described in further detail hereinbelow with reference to FIGS. 2A-C and 3A-3E.

Reference is now made to FIGS. 2A, 2B and 2C, which are schematic illustrations of views operational head 40 disposed at distal portion 34 of delivery tool 30, in accordance with some applications of the present invention. As shown, operational head 40 comprises an expandable element 50, an implant 70, and a cutting electrode 60, in accordance with some applications of the present invention. Operational head 40 is shown in FIGS. 2A, 2B and 2C in an expanded operational state thereof.

### Expandable Element

Expandable element 50 is typically expanded within the urethra such as to enlarge the urethra in the area to be treated. For example, expandable element 50 comprises an inflatable balloon. For some applications, expandable element 50 is a self-expanding device that is configured to self-expand upon being released from the delivery tool. For some applications, expandable element 50 is configured to be expanded and/or collapsed (inflated and/or deflated in case of a balloon) as desired by the user. In some applications, expandable element 50 is made of one or more materials, for example: PET, nylon, silicon, latex, polyurethane, and/or Pebax®. In some applications, expandable element 50 has an internal volume of from about 2 cc to about 10 cc, e.g., 5 cc. Optionally from about 1 cc to about 15 cc, e.g., 3-4 cc, e.g., 3-7 cc. Optionally from about 10 cc to about 30 cc. In some applications, the diameter of the expandable element 50 is from about 10 mm to about 20 mm. Optionally from about 5 mm to about 35 mm. Optionally from about 3 mm to about 50 mm. In some applications, expandable element 50 can be inflated to an internal pressure of from about 1 atm to about 20 atm. Optionally from about 0.5 atm to about 30 atm. Optionally from about 5 atm to about 50 atm. In some applications, expandable element 50 is in communication with inlet/outlet 28 in order to facilitate inflation and deflation of expandable element 50.

For some applications, expandable element 50 comprises a transparent, or partially transparent, expandable element, e.g., a transparent balloon, which is configured to facilitate visualization by an imaging device of the area of the urethra that is treated via the expandable element.

For some applications, expandable element 50 does not comprise a balloon. For some such applications, expandable element 50 typically comprises a mechanical dilator configured expanded within the urethra such as to enlarge the urethra in the area to be treated, e.g., an umbrella shaped dilator.

### Implant

Implant 70 is disposed on an outer surface of expandable element 50 and is configured to be implanted within the wall of the urethra (i.e., within tissue surrounding the lumen of the urethra) to maintain the urethra in a dilated state to overcome constriction of the urethra due to the enlarged prostate.

Implant 70 is configured to be delivered to the constricted location in the urethra in a spiral configuration, being wound around the radially-collapsed expandable element. When the distal portion of the delivery tool reaches the location in the urethra that is to be treated, the expandable element 50 is expanded, causing implant 70 to unwind into a C-shaped open ring (for example, an omega-shaped, that is disposed on the outer surface of expandable element 50, as shown in FIGS. 2A-C. Typically, the C-shaped open ring implant has a flat surface.

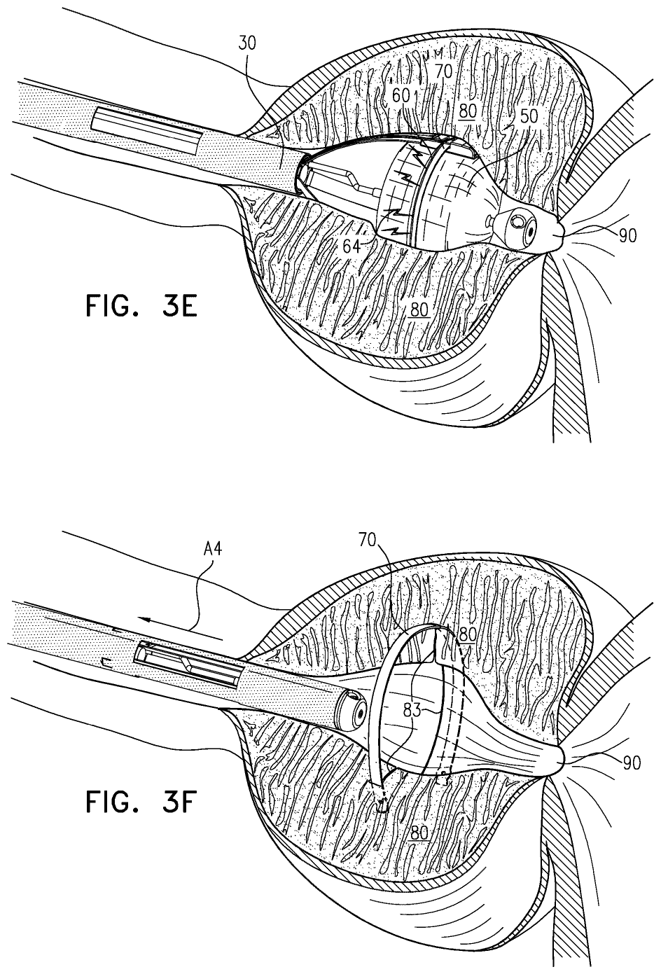

For some applications, when expandable element 50 is expanded and is unconstrained by surrounding tissue, implant 70 unwinds into the configuration shown for example in FIGS. 5A-B, in which, implant 70 is shaped to define a substantially straight middle section disposed between two curved end sections on either side of the middle section. However, when released within the urethra, the implant typically assumes a generally-similar shape to that shown in (and described with reference to) FIGS. 2A-C, because the middle section is prevented from straightening, due to end of the implant being constrained by surrounding tissue.

For some applications, implant 70 comprises one or more of: stainless steel, titanium, PET, PEEK, and/or PA. For some applications, implant 70 comprises a resilient material such as a shape-memory material, e.g., nitinol. For some applications, implant 70 is coated with a biocompatible electrically insulating coating, e.g., a parylene coating.

In some applications, implant 70 has a length of 30 mm to 100 mm when unconstrained, e.g., 40 mm to 80 mm, e.g., 50 mm to 70 mm.

In some applications, implant 70 has a thickness ranging from 0.0 1 mm to 1 mm, e.g., 0.1 mm to 0.8 mm, e.g., 0.3 to 0.6 mm, e.g., 0.1 mm, 0.25 mm, 0.3 mm, or 0.5 mm. Typically, thin implant 70 is better suited for being implanted deeper in the tissue and facilitating improved healing, compared to a thicker implant.

In some applications, implant 70 has a width of 0.5 mm to 4 mm, e.g., 0.7 mm to 2 mm, e.g., 1 mm to 1.5 mm, e.g., 0.5 mm, 0.7 mm, 1 mm, 1.3 mm, 2 mm, 3 mm. In some applications, having a relatively small width allows implant 70 to be inserted deeper in tissue compared to a wider implant. Additionally, or alternatively, tissue around the implant is allowed to heal better around an implant with a relatively small width.

In some applications, the forces applied by implant 70 to tissue of the urethra are 25 gr to 500 gr, e.g., 50 gr to 100 gr, e.g., 40 gr to 150 gr, e.g., 50 gr, 60 gr, 70 gr, 100 gr, 120 gr.

In some applications, implant 70 is configured to remain in a subject's body for a predetermined period of time, after which time, implant 70 is removed from the body. For example, implant 70 is configured to remain in the subject's body for 3 months, 6 months, 12 months, 36 months or longer. Alternatively, implant 70 is formed of a biodegradable material. In some such applications, implant 70 biodegrades over a period of time chosen by the physician, for example after 3 months, 6 months, 12 months or 36 months. Alternatively, implant 70 is configured to remain in a subject's body for longer periods of time.

### Cutting Electrode

Operational head 40 of apparatus 20 further comprises a cutting electrode 60 that is configured to create an implant-receiving cut in the walls of the urethra in order to create an area shaped and sized to accommodate implant 70. Cutting electrode 60 applies electrical current (e.g., diathermic electrical current) to tissue of the urethra to form the implant-receiving cut in the tissue surrounding the lumen of the urethra by the application of the electrical current. Typically, implant 70 is electrically insulated from cutting electrode 60.

As shown in FIGS. 2A-C, cutting electrode 60 is typically disposed on an outer surface of implant 70 (the surface of implant 70 that faces the wall of the urethra). When expandable element 50 expands in the urethra, and implant 70 opens into the C-shaped open ring configuration, cutting electrode 60 is positioned on implant 70 such that electrode 60 contacts the tissue of the urethra such that when electrical current is applied through cutting electrode 60, the implant-receiving cut is formed in the tissue of the urethra.

Cutting electrode 60 typically comprises a static electrode (i.e., an electrode that is configured to form the implant-receiving cut without being moved). As shown in FIGS. 2A-C, cutting electrode 60 is shaped to conform with the shape of implant 70 such that the implant-receiving cut is formed in the shape of implant 70 substantially without movement, e.g., rotation, of the cutting electrode. For some applications, by being configured to form the implant-receiving cut without being moved, damage to the surrounding tissue is reduced or prevented, relative to if the electrode (and/or additional parts of the operational head) was to require movement, e.g., rotation in order to form the implant-receiving cut.

For some applications, cutting electrode 60 forms the implant-receiving cut while cauterizing the tissue by application of the electrical current. Thus, application of the electrical current by cutting electrode 60, creates the implant-receiving cut in the tissue surrounding the lumen of the urethra, and at the same time causes cauterization of the tissue. As a result, bleeding from the tissue that may be caused by creating the cut in the tissue, is typically reduced or prevented. Additionally, in the event of bleeding from the cut in the tissue, the heat generated by application of the electrical current by cutting electrode 60, typically causes quick coagulation of the blood, thereby further ensuring reduced or prevention of bleeding during use of apparatus 20.

For some applications, implant 70 is deployed while the implant-receiving cut is being made by cutting electrode 60, or after the cut is completed. Typically, the implant is deployed in the wall of the urethra and also at least partially embedded through the wall of an enlarged portion of the prostate surrounding a constricted portion of the urethra.

Typically, cutting electrode 60 is releasably coupled to implant 70, and is configured to be retracted from the urethra subsequently to forming the cut. Cutting electrode 60 is typically pulled proximally until it is removed from the urethra and from the subject's body. For some applications, a proximal extension from the electrode (such as a string or a wire) is disposed in delivery tool 30, such that cutting electrode 60 is configured to be retracted from the urethra by pulling the proximal extension from the electrode in a proximal direction.

In some applications, cutting electrode 60 is configured to create the implant-receiving cut in the tissue having a depth of 1 mm-20 mm, e.g., 4 mm-10 mm, e.g., 2 mm-8 mm, e.g., 3 mm, 5 mm, 7 mm, 10 mm, 12 mm, or 15 mm.

For some applications, as will be described in further detail hereinbelow, cutting electrode 60 is releasably coupled to implant 70 using one or more sutures (or any type of securing wire). Typically, the cutting electrode is decoupled from the implant by burning of the sutures when electrical current is applied by the cutting electrode.

For some applications, in addition to, or alternatively to cutting electrode 60, implant 70 or a portion thereof, is configured to apply electrical current to the tissue to form the cut in the tissue of the urethra. For some such applications, both cutting electrode 60 and implant 70 apply electrical current to the tissue surrounding the lumen of the urethra to form an implant-receiving cut in the tissue. Alternatively, apparatus 20 does not comprise a cutting electrode 60, rather, implant 70 creates the implant-receiving cut in the wall of the urethra by application of the electrical current to tissue surrounding the lumen of the urethra to form an implant-receiving cut in the tissue, and to be deployed in the cut while the cut is being made.

Reference is again made to FIGS. 1A-B and FIGS. 2A-C. Below is a description of examples of additional (possibly optional) elements of apparatus 20.

Implant Carrier and/or Implant Holders

For some applications, apparatus 20 comprises additional elements configured to facilitate delivery and release of implant 70 from the outer surface of expandable element 50 at the chosen location. For example, apparatus 20 comprises one or more implant carrier arms 74 and/or implant holders 72. Implant carrier arm 74 is typically coupled to shaft 36 and to implant 70 and disposed outside the outer surface of the expandable element. Implant carrier arms 74 are typically configured to release the implant into the implant-receiving cut subsequently to, or during, the cut being formed by the cutting electrode.

Implant holders 72 are typically configured to restrict detachment of implant 70 from expandable element 50 and insertion of the implant into the urethra wall. Implant 70 is disposed between the implant carrier arm and the implant holder such that the implant is held in place by the implant carrier arm and the implant holder. When it is desired to deploy implant 70 in the tissue, implant holders 72 are retracted in a proximal direction, allowing implant 70 to be implanted in the tissue subsequently to, or during, the implant-receiving cut being formed by cutting electrode 60, as described hereinabove. Implant carrier arms 74 and/or implant holders 72 are typically coated with a biocompatible electrically insulating coating, e.g., a parylene coating. For some applications, implant holders 72 are made of a biocompatible plastic polymer.

### Imaging Device

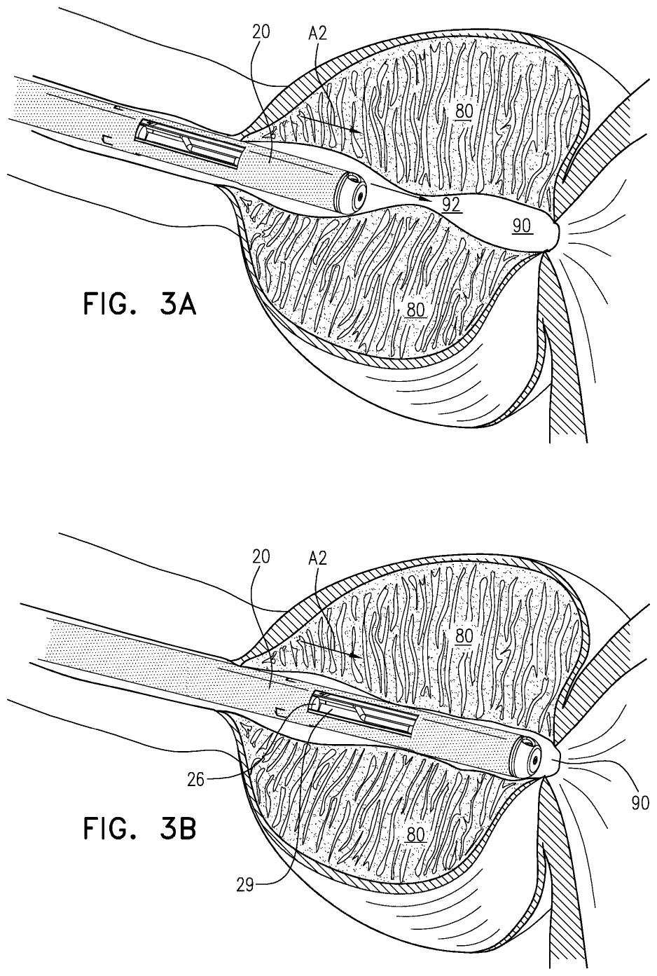

For some applications, apparatus 20 further comprises an imaging device (a camera, or an optical apparatus) disposed in the delivery tool and configured to provide visualization of the area of the urethra that is to be treated and/or of the implantation procedure. Typically, an imaging device 26 (such as a camera and/or an optical fiber) is incorporated into apparatus 20. FIG. 1A shows the proximal end of imaging device 26 that is incorporated into apparatus 20. Imaging device 26 typically extends from the proximal portion 32 of delivery tool 30 until the vicinity of expandable element 50, e.g., slightly proximally to the expandable element (as shown for example in FIGS. 3B-C). As described hereinabove, expandable element 50 is typically transparent (or partially transparent), thereby providing visualization of the treated area in the urethra via the expandable element. For some applications, imaging device 26 is configured to provide a distally facing view, and/or a lateral facing view through apertures 29 in delivery tool 30.

In some applications, imaging device 26 has a field of view of 0 degrees (i.e., a distally facing view) to about ±90 degrees, optionally from about 0 degrees to about ±70 degrees, optionally from about 0 degrees to about ±120 degrees. Typically, imaging device 26 is configured to allow the user to navigate through the urethra, visualize the location of the constriction of the urethra, and place implant 70 under visualization at the desired location.

Reference is now made to Figs. FIGS. 3A, 3B, 3C, 3D, 3E and 3F, which are schematic illustrations of apparatus 20 for treatment of the urethra being advanced and deployed in the urethra to dilate a constricted area in the urethra, in accordance with some applications of the present invention.

FIG. 3A and FIG. 3B show apparatus 20 being advanced distally within urethra 90 toward an area 92 in the urethra which is to be treated due to area 92 being constricted by enlargement of prostate 80. The direction of distal advancement of apparatus 20 is indicated by arrow A2. When the distal portion of delivery tool 30 reaches the constricted location in the urethra, a portion of delivery tool 30 is retracted proximally (in the direction indicated by arrow A3), thereby exposing operational head 40 (FIG. 3C). Typically, during distal advancement through the urethra in delivery tool 30, expandable element 50 is in a collapsed state and implant 70 is wound around the collapsed expandable element. Cutting electrode 60 is typically disposed around the outer surface of implant 70.

As shown in FIGS. 3D and 3E, once operational head 40 is exposed, expandable element 50 is allowed to expand (e.g., by self-expanding and/or by inflation through an inflation lumen 37). As shown, in FIG. 3D, for some applications, expandable element 50 comprises an inflatable balloon 50. The inflatable balloon is inflated within the urethra thereby enlarging the urethra in the area to be treated, and radially expanding implant 70 from the configuration shown in FIG. 3C, in which implant 70 is wound around the expandable element, into a C-shaped open ring configuration (e.g., an omega-shape, shown in FIGS. 3D and 3E. FIG. 3D shows initiation of inflation of the inflation balloon, and FIG. 3E shows the inflation balloon in a fully inflated state to dilate the urethra. As described hereinabove, typically, the C-shaped open ring implant has a flat surface.

Implant 70 is disposed on the outer surface of inflatable balloon and cutting electrode 60 is disposed on the outer surface of implant 70. Cutting electrode 60 applies electrical current to the tissue to form the cut in the tissue (the electrical current is indicated in FIG. 3E by reference numeral 64). As further shown in FIG. 3E, the shape of cutting electrode 60 conforms to the shape of implant 70 such that cutting electrode 60 creates a cut in the shape of implant 70, without moving, e.g., rotating the electrode. In other words, cutting electrode 60 remains static while it applies electrical current to the tissue to form the cut in the tissue. By virtue of the cutting electrode having a shape that conforms to the shape of implant 70, the implant receiving cut is formed in the shape of implant 70 substantially without rotation of the cutting electrode. For some applications, by being configured to form the implant-receiving cut without being moved, damage to the surrounding tissue is reduced or prevented, relative to if the electrode (and/or additional parts of the operational head) was to require rotation in order to form the implant-receiving cut.

For some applications subsequently to, or during, the cut being formed by the cutting electrode, the portion of the implant that is held by the one or more implant carrier arms 74 and/or implant holders 72 (which is typically the central portion of the implant) is released from the outer surface of expandable element 50, thereby allowing implant 70 to be implanted in the tissue. For example, the portion of the implant that is held by the one or more implant carrier arms 74 and/or implant holders 72 is released by retracting the implant holders 72 in a proximal direction, as described hereinabove.

As noted above, for some applications, expandable element 50 is transparent. The transparency of the expandable element typically facilitates visualization of the location of the constriction of the urethra by imaging device 26 even at the stage shown in FIG. 3D (when the expandable element is being expanded at the location of the constriction).

FIG. 3F shows implant 70 deployed through the wall of the urethra in tissue of prostate 80. As shown, implant 70 maintains the urethra in a dilated state in the area which was previously constricted by the prostate. In FIG. 3F, cutting electrode 60 has already been retracted following formation of the implant-receiving cut (indicated by reference numeral 83). Additionally, the expandable element has collapsed (e.g., by the balloon being deflated). FIG. 3F shows the delivery tool being retracted proximally in the direction of arrow A4. (It is noted that although FIG. 3F shows operational head 40 fully retracted into shaft 36 when the delivery tool is retracted proximally, it may be the case that the delivery tool is retracted proximally without while operational head 40 is in a collapsed state, but not retraced inside shaft 36). Typically, implant 70 remains embedded in tissue of the urethra wall and/or the prostate to maintain the urethra in a dilated state, relieving the symptoms of BPH.

As noted hereinabove, for some applications, when expandable element 50 is expanded and is unconstrained by surrounding tissue, implant 70 unwinds into the configuration shown for example in FIGS. 5A-B, in which, implant 70 is shaped to define a substantially straight middle section disposed between two curved end sections on either side of the middle section. However, when released within the urethra (e.g., both in the step shown in FIG. 3E and in the step shown in FIG. 3F), the implant typically assumes a generally-similar shape to that shown in (and described with reference to) FIGS. 2A-C, because the middle section is prevented from straightening, due to end of the implant being constrained by surrounding tissue.

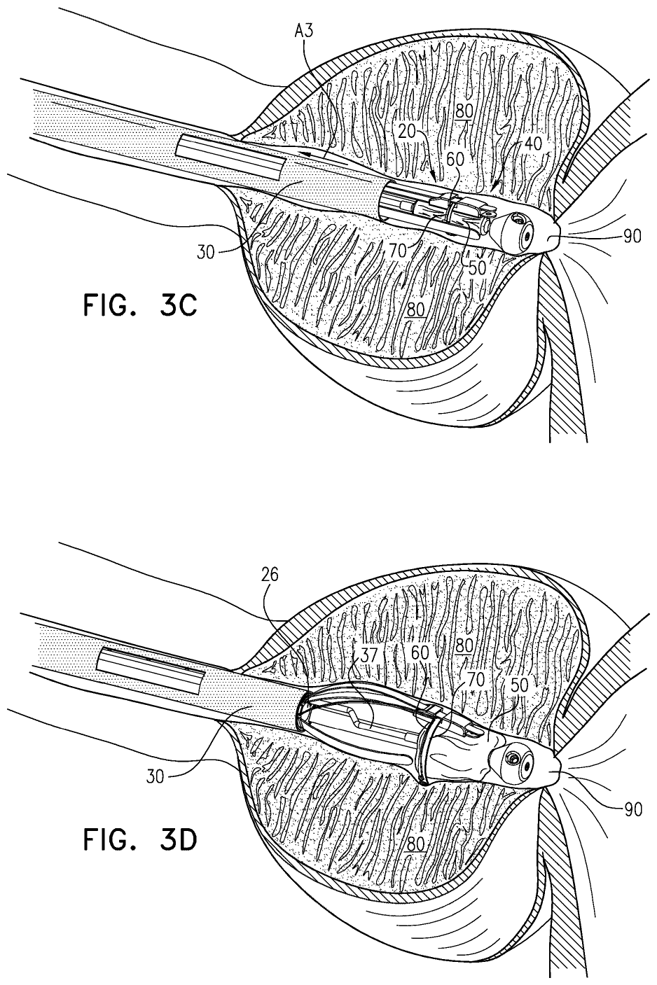

Reference is made to FIG. 4, which is a flow chart showing steps of a method for treatment of a urethra performed in accordance with some applications of the present invention. For some applications, the apparatus comprising an expandable element (e.g., an inflatable element), an implant and a cutting electrode are distally advanced within the urethra to an area restricted by an enlarged prostate (step 110). The urethra is expanded at the constricted site by inflating the expandable element (step 120). Typically, subsequently, the cutting electrode forms a cut in the wall of the urethra by application of electrical current (e.g., diathermic electric current) through the electrode, while keeping the electrode static substantially without rotating the electrode (step 130). The implant is released into the cut (step 140) to maintain the urethra in the dilated state (step 150). Following release of the implant the delivery tool is maneuvered in the urethra in a proximal direction until complete removal of the device.

Reference is still made to FIG. 4. For some applications, prior to distally advancing apparatus 20 within the urethra (step 110), a length of the enlarged prostate is measured, and accordingly, a center of the constricted area in the urethra (the stricture in the urethra being due to the enlarged prostate), is estimated. Apparatus 20 is then positioned in the urethra based on the measurements of the prostate, and the estimated location of the stricture.

Reference is now made to FIGS. 5A-B, which are schematic illustrations of components of operational head 40 disposed at the distal portion of the apparatus for treatment of a urethra, in accordance with some applications of the present invention. As described hereinabove, for example with reference to FIG. 2, operational head 40 comprises expandable element 50, implant 70, and cutting electrode 60.

FIG. 5A shows operational head 40 with expandable element 50 in a deflated state, and implant 70 prior to release into the tissue. (It is noted that the configuration shown in FIG. 5A is shown for illustrative purposes. When in actual use, implant 70 is typically wound around a deflated expandable element 50, or assumes a generally C-shaped open ring configuration (for example, an omega-shape) due to being constrained by tissue, as described hereinabove.)

FIG. 5B shows operational head 40 in an operational state comprising expandable element 50 in the expanded state thereof, implant 70, and cutting electrode 60. It is noted that both FIGS. 5A and 5B show the implant in the absence of surrounding tissue. In the configuration shown in FIGS. 5A-5B, the implant is shaped to define a substantially straight middle section disposed between two curved end sections on either side of the middle section, as described hereinabove. However, when the implant is released into the urethra, using the techniques described herein, the implant typically assumes an expanded C-shape (e.g., an omega shape), due to pressure of the surrounding tissue preventing the implant from fully assuming its unconstrained configuration.

As shown in FIGS. 5A and 5B, for some applications, cutting electrode 60 is releasably secured to implant 70 by one or more sutures 65. Typically, one or more sutures 65 maintain cutting electrode 60 coupled to implant 70 and positioned on the outer surface of implant 70 during delivery and radial expansion of implant 70, prior to application of electrical current through cutting electrode 60. When electrical current is applied through cutting electrode 60, sutures 65 are decoupled from cutting electrode 60 (typically by being dissolved/burnt by heat generated by the electrical current), and the implant-receiving cut is formed in the tissue of the urethra. When the implant-receiving cut is formed in the tissue of the urethra, implant 70 is deployed in the cut through the wall of the urethra and in tissue of the prostate. As described hereinabove, typically, cutting electrode 60 is retracted and removed from the body following formation of the implant-receiving cut. For some applications, sutures 65 are made of a biocompatible and bioabsorbable material such that any remaining portions of the sutures following application of the electrical current by cutting electrode 60, are absorbed into the body of the subject. Alternatively, sutures 65 are non-absorbable and thus are not absorbed by the body. Generally, any portions of the sutures (either absorbable or non-absorbable) that remain following application of the electrical current by cutting electrode 60, are extracted from the urethra together with other portions of apparatus 20 (e.g., using a grasper or any other suitable tool). For some applications, a plurality of sutures 65 are connected to each other by a connecting wire/suture such that any remaining sutures following application of the electrical current by the cutting electrode can be removed together with a single extraction.

Alternatively, or additionally, cutting electrode 60 is releasably coupled to implant 70 by other types of coupling techniques and devices. For example, cutting electrode 60 may be releasably coupled to implant 70 by using a mechanical coupling element (e.g., a clip), and or an adhesive (e.g., glue).

Reference is still made to FIGS. 5A-B. As shown, for some applications, cutting electrode 60 has coiled tips 62 (resembling one or more turns of a coil spring). Under normal use, each one of coiled tips 62 typically holds a respective end of cutting electrode 60 in place with respect to implant 70 by preventing the end of the cutting electrode from passing through the suture that is disposed closest to that end of the implant. On the other hand, in the event that one or more of sutures 65 are not decoupled from cutting electrode 60 by the application of electrical current via the cutting electrode (e.g., due to a malfunction of the cutting electrode) and apparatus 20 therefore requiring removal (and optional redeployment), coiled tips 62 allow for simple and safe retraction of cutting electrode 60 by the coiled tips being actively pulled through one or more of sutures 65, such that the coiled tips straighten and pass through the one or more sutures. In this respect, the coiled tips allow for removing the cutting electrode from urethra compared to if the tips of cutting electrode 60 were secured to the implant with a clamping device, by way of example. Typically, in the event of the aforementioned malfunction, once the ends of the cutting electrode have been pulled through the one or more sutures, cutting electrode 60 is retracted by being pulled in a proximal direction until its complete removal from the body of the subject.

Reference is again made to FIGS. 2A-C and FIGS. 5A-B. As described hereinabove with reference to FIGS. 2A-C, apparatus 20 comprises one or more implant carrier arm 74, which are coupled to shaft 36 and implant 70. Implant carrier arms 74 typically extend from shaft 36 to contact and support implant 70, as shown in FIG. 2. Additionally, in some applications, the implant carrier arms assist in maintaining the urethra in a dilated state by applying pressure to the walls of the urethra in an area that is proximal to expandable element 50 (shown for example in FIGS. 3D and 3E).

For some applications, apparatus 20 comprises 2-4, e.g., 3, implant carrier arms 74. Alternatively, apparatus 20 comprises fewer implant carrier arms 74. For example, as shown in FIG. 5A, apparatus 20 comprises only a single implant carrier arm 74 extending from shaft 36 to contact implant 70. Reducing the number of implant carrier arms 74 typically simplifies delivery and release of implant 70 into the cut formed in the urethra. Additionally, or alternatively, a potential advantage of reducing the number of implant carriers arms 74, is reducing metal-on-metal contacts between implant carrier arms 74 and implant 70.

As further shown in FIG. 5A, for some applications, apparatus 20 additionally comprises one or more radial arms 76. In applications in which apparatus 20 comprises a reduced number of implant carrier arms 74 that reach implant 70 (e.g., a single implant carrier arm 74), apparatus 20 typically comprises one or more radial arms 76 that assist in maintaining the urethra in a dilated state by applying pressure to the walls of the urethra in an area that is proximal to expandable element 50. Radial arms 76 typically have a length that is sufficient to apply pressure to walls of the urethra proximally to expandable element 50 when the expandable element is expanded to overcome the constriction of the urethra. However, radial arms 76 are not sufficient in length to reach and contact implant 70, thereby preventing potential metal-on-metal contacts between radial arms 76 and implant 70. Typically, by applying pressure to the walls of the urethra in an area that is proximal to expandable element 50, the walls dilate this area such that the area is visible to imaging device 26.

It is noted that in general apparatus 20 is structured to avoid voltage breakdown. For example, by reducing metal-on-metal contacts between components of apparatus 20, and by providing metallic components with insulating coatings and/or replacing metal components with plastic. For example, as described hereinabove, implant carrier arm 74 and/or implant holders 72 are typically coated with a biocompatible electrically insulating coating, e.g., a parylene coating. Alternatively or additionally, implant holders 72 are made of a biocompatible electrically non-conductive polymer, e.g., a biocompatible plastic.

Reference is made to FIGS. 1A-5B. Typically, apparatus 20 is controlled by a user through user interface elements (e.g., knobs, buttons, and/or levers), typically located in the handle, which either deliver a manual motion or activate motors (e.g., linear and/or rotary motors) also typically located in the handle. As described herein, the implant-receiving cut is formed in tissue of the urethra, while the cutting electrode is kept static. In other words, the implant-receiving cut is formed generally in the shape of the implant substantially without movement, e.g., rotation of the electrode and/or the implant. A static electrode, compared to a moving electrode, allows apparatus 20 to be operated in a partially or fully mechanical manner thereby avoiding the need for sophisticated software and/or hardware to control and synchronize release of the implant with movement of the cutting electrode. In general, it is noted that apparatus 20 is configured to be operated in a fully mechanical manner.

### Experimental Data

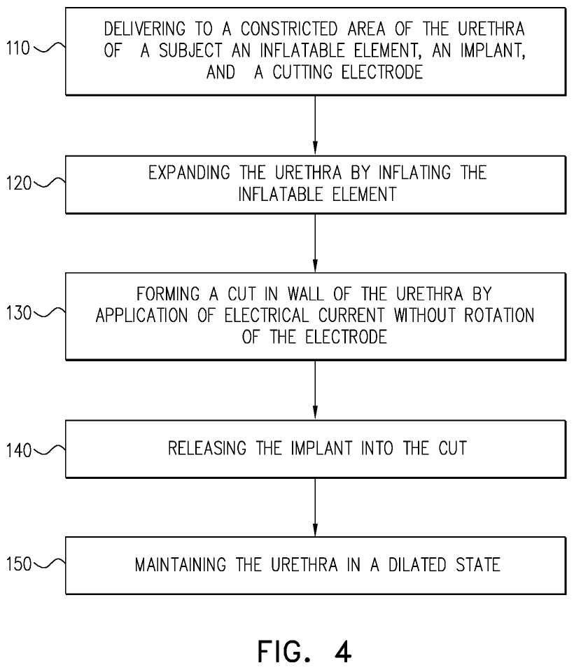

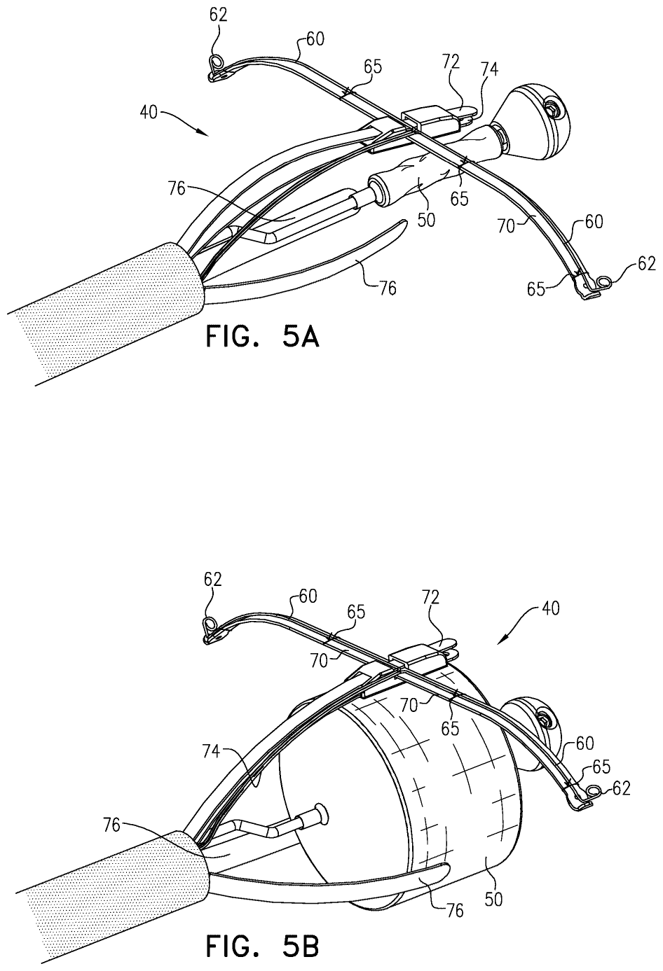

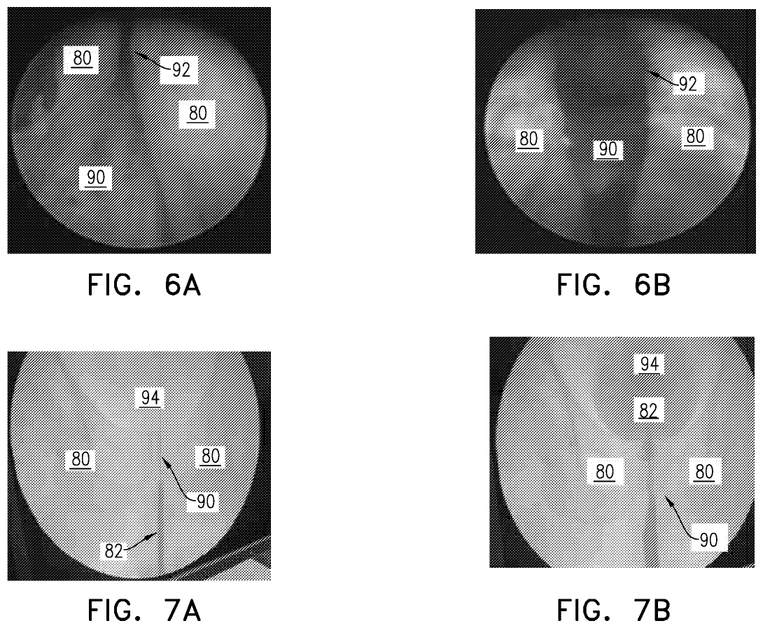

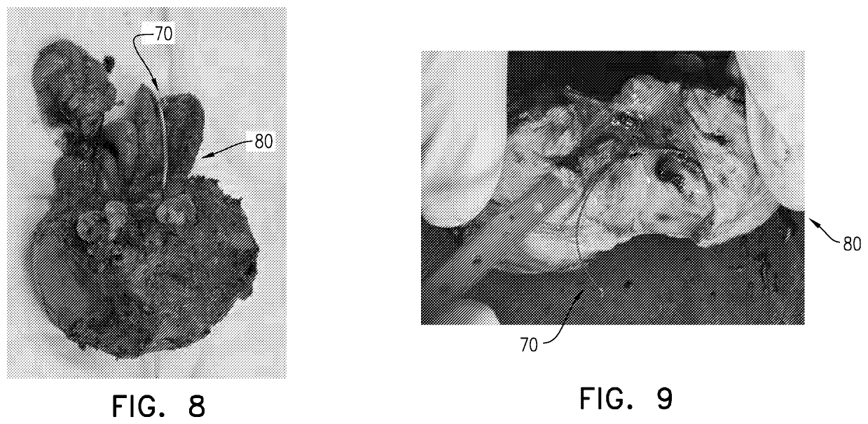

Reference is now made to FIGS. 6A-B, FIGS. 7A-B, FIG. 8 and FIG. 9, which are photographs of experimental data obtained in accordance with some applications of the present invention. More specifically, the experiments described hereinbelow with reference to FIGS. 6A-B, FIGS. 7A-B, FIG. 8 and FIG. 9 were performed by the inventors in accordance with applications of the present invention and using apparatus 20 and techniques described herein.

Reference is first made to FIGS. 6A and 6B, which are photographs showing an example of a urethra of a subject, prior to treatment (FIG. 6A), and following treatment (FIG. 6B) of the subject using apparatus 20 for treatment of a urethra that is constricted due to benign prostatic hyperplasia (BPH), in accordance with some applications of the present invention. FIG. 6A shows urethra 90 having constricted area 92 due to enlarged prostate 80. FIG. 6B shows prostate 80 following treatment with apparatus 20 in accordance with the techniques for use of apparatus 20 described herein. As shown in FIG. 6B, following use of apparatus 20, the obstruction of the urethra is overcome such that area 92 of the urethra is no longer constricted.

Reference is now made to FIGS. 7A and 7B, which are photographs showing a contrast fluid being injected through a urethra of a subject following treatment of the subject using apparatus 20 and techniques described herein for treatment of a urethra that is constricted due to benign prostatic hyperplasia (BPH), in accordance with some applications of the present invention. FIG. 7A shows urethra 90 following treatment with apparatus 20 and prior to the contrast fluid 82 being injected into urethra 90. FIG. 7B shows contrast fluid 82 being injected through urethra 90 and reaching bladder 94 indicating unobstructed passage through urethra 90 resulting from the treatment with apparatus 20.

Reference is now made to FIG. 8 and FIG. 9, which are photographs showing implant 70 implanted in an enlarged prostate, in accordance with some applications of the present invention. As described hereinabove, with reference to FIGS. 1A-5B, implant 70 is implanted in the prostate to maintain the urethra in a dilated state. As shown in FIG. 8 and FIG. 9, implant 70 is fully embedded in prostate 80 such that it does not interfere, and is not exposed, to the flow of urine through the urethra. As shown, implant 70 is shaped to define an omega shaped implant that is deployed typically in the center of the prostate and is embedded with tissue of the prostate at a depth of 2.5 mm-5 mm (FIG. 9).

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for treatment of a urethra, a lumen of which is constricted, the apparatus comprising:
a delivery tool having a proximal portion and a distal portion, the distal portion configured to be advanced to a location in an area of the urethra that is to be treated, the distal portion comprising:
an expandable element that is configured to be expanded within the urethra such as to enlarge the urethra in the area to be treated, the expandable element defining an outer surface;
an implant that is disposed outside the outer surface of the expandable element; and
a cutting electrode releasably coupled to an outer surface of the implant and configured to apply electrical current to tissue of the urethra to form an implant-receiving cut in tissue surrounding the lumen of the urethra by the application of the electrical current,
wherein the cutting electrode is shaped to conform with a shape of the implant such that the implant-receiving cut is formed in the shape of the implant substantially without movement of the cutting electrode.

2. The apparatus according to claim 1, wherein the cutting electrode is shaped to conform with a shape of the implant such that the implant-receiving cut is formed in the shape of the implant substantially without rotation of the cutting electrode.

3. The apparatus according to claim 1, wherein the cutting electrode is configured to be retracted from the urethra subsequently to forming the implant-receiving cut.

4. The apparatus according to claim 1, wherein the cutting electrode has a distal portion that is releasably coupled to the implant and a proximal extension that is disposed in the delivery tool, and wherein the cutting electrode is configured to be retracted from the urethra by pulling the proximal extension of the electrode in a proximal direction.

5. The apparatus according to claim 1, wherein the implant is configured as a C-shaped open ring.

6. The apparatus according to claim 1, wherein the implant is configured to be wound around the expandable element, while the distal portion of the delivery tool is advanced to the location in an area of the urethra that is to be treated.

7. The apparatus according to claim 1, wherein the implant has a thickness of 0.01-1 mm.

8. The apparatus according to claim 1, wherein the implant has a width of 0.5-4 mm.

9. The apparatus according to claim 1, wherein the cutting electrode is formed of nitinol.

10. The apparatus according to claim 1, wherein the implant is electrically insulated from the cutting electrode.

11. The apparatus according to claim 1, wherein the implant is configured to maintain the urethra in a dilated state in the area of the cut by being deployed in the cut.

12. The apparatus according to claim 1, further comprising an imaging device disposed in the delivery tool and configured to provide visualization of the area of the urethra that is treated by the imaging device.

13. The apparatus according to claim 1, wherein the expandable element comprises an inflatable balloon.

14. The apparatus according to claim 13, wherein the inflatable balloon is transparent.

15. The apparatus according to claim 14, further comprising an imaging device disposed in the delivery tool, and wherein the inflatable balloon is configured to provide visualization of the area of the urethra that is treated by the imaging device, via the inflatable balloon.

16. The apparatus according to claim 1, further comprising one or more sutures configured to releasably couple the cutting electrode to the implant prior to application of the electrical current by the cutting electrode.

17. The apparatus according to claim 16, wherein the one or more sutures are configured to become decoupled from the implant by the application of the electrical current, to thereby release the cutting electrode from the implant.

18. The apparatus according to claim 16, wherein the cutting electrode comprises coiled tips at each end of the cutting electrode, wherein each one of the coiled tips holds a respective end of the cutting electrode in place with respect to the implant by preventing the end of the cutting electrode from passing through one of the sutures that is disposed closest to that end of the implant, and wherein in response to the cutting electrode being pulled proximally, the coiled tip is configured to straighten and pass through the one or more sutures.

19. The apparatus according to claim 1, further comprising a single implant carrier arm extending from a shaft of the delivery tool and being disposed outside the outer surface of the expandable element, the implant carrier being configured to release the implant into the implant-receiving cut subsequently to the cutting electrode forming the implant-receiving cut.

20. The apparatus according to claim 19, wherein the implant carrier arm is coated with an electrically insulating coating.

21. The apparatus according to claim 19, further comprising an electrically-non-conductive implant holder coupled to the implant carrier arm, and wherein:
the implant is configured to be disposed between the implant carrier arm and the implant holder such that the implant is held in place by the implant carrier arm and the implant holder; and
the implant holder being configured to be retracted proximally with respect to the implant carrier arms such that the implant is released by the implant carrier arms into the implant-receiving cut.

22. The apparatus according to claim 19, further comprising at least one radial arm having a length which is: (a) sufficient to apply pressure to the walls of the urethra to dilate the urethra in an area that is proximal to the expandable element, when the expandable element is in an expanded state; and (b) not sufficient to contact the implant.

23. A method for treating a urethra, a lumen of which, is constricted due to benign prostatic hyperplasia (BPH) comprising:
identifying a constricted area of the urethra requiring treatment;
inserting into the urethra a delivery tool that includes:
an expandable element that defines an outer surface,
an implant disposed on the outer surface of the expandable element, and
a cutting electrode releasably coupled to an outer surface of the implant;
using the delivery tool, delivering, to the identified constricted area of the urethra, the inflatable element;
expanding the urethra by expanding the expandable element in the identified constricted area of the urethra;
subsequently, forming an implant-receiving cut in the tissue surrounding the lumen of the urethra, by applying an electrical current to the tissue using the cutting electrode, wherein the cutting electrode is shaped to conform with a shape of the implant such that the cutting electrode forms the implant-receiving cut in the shape of the implant substantially without the cutting electrode being moved; and
releasing the implant into the cut to maintain the urethra in a dilated state.

24-27. (canceled)
